SEQUENCE LISTING (20 sequences)

SEQ ID NO: 1 - protein, 8 aa, synthetic construct
GFTFTNYY

SEQ ID NO: 2 - protein, 10 aa, synthetic construct
IRDKANGYTT

SEQ ID NO: 3 - protein, 13 aa, synthetic construct
ARVLRYLGYNFDY

SEQ ID NO: 4 - protein, 6 aa, synthetic construct
QDIGNF

SEQ ID NO: 5 - protein, 9 aa, synthetic construct
QQDNTLPWT

SEQ ID NO: 6 - protein, 122 aa, synthetic construct
EVKLVESGGGLVQPGGSLRLSCATSGFTFTNYYMSWVRQPPGKALEWLAFIRDKANGYTTEYSASVKGRFTISRDNSQNILYLQMNTLRAEDSATYYCARVLRYLGYNFDYWGQGTTLTVSS

SEQ ID NO: 7 - protein, 107 aa, synthetic construct
DIQMTQTTSSLSASLGDRVTISCRASQDIGNFLTWYQQKPDGTVKLLIYYRSRLHSGVPSRFSGSGSGTDYSLTISNLEQEDIATYFCQQDNTLPWTFGGGTKLEIK

SEQ ID NO: 8 - protein, 122 aa, synthetic construct
EVQLVESGGGLVQPGGSLRLSCAASGFTFTNYYMSWVRQAPGKGLEWVAFIRDKANGYTTEYSASVKGRFTISRDNSKNTLYLQMNSLRAEDTAVYYCARVLRYLGYNFDYWGQGTLVTVSS

SEQ ID NO: 9 - protein, 107 aa, synthetic construct
DIQMTQSPSSLSASPGDRVTISCRASQDIGNFLTWYQQKPGQAPKLLIYYRSRLHSGVPSRFSGSGSGTDYTLTISSLEPEDFATYYCQQDNTLPWTFGQGTKVEIK

SEQ ID NO: 10 - protein, 15 aa, synthetic construct
GGGGSGGGGSGGGGS

SEQ ID NO: 11 - protein, 5 aa, synthetic construct
GGGGS

SEQ ID NO: 12 - DNA, 747 bp, synthetic construct
  source 1..747: mol_type = other DNA
gacatccagatgacccagtccccatccagcctgtccgccagcccaggcgacagagtgaccatctcttgccgcgcttcccaggacatcggcaacttcctgacatggtaccagcagaagcccggccaggcccctaagctgctgatctactataggagccggctgcattctggcgtgccctccaggttttctggctccggcagcggcaccgattataccctgacaatctcttccctggagcctgaggacttcgctacatactattgccagcaggataataccctgccatggacatttggccagggcacaaaggtggagatcaagggaggaggaggatccggaggaggaggaagcggcggcggcggctctgaggtgcagctggtggagtccggaggaggcctggtgcagccaggaggcagcctgagactgtcttgtgccgcttccggcttcacctttacaaactactatatgagctgggtgaggcaggctcctggcaagggcctggagtgggtggccttcatccgggacaaggctaatggctacaccacagagtattctgcttccgtgaagggcaggtttaccatcagccgggacaactctaagaatacactgtacctgcagatgaactctctgcgcgccgaggataccgccgtgtactattgtgctagagtgctgcgctacctgggctataattttgattattggggccagggcaccctggtgacagtgagctctggaggaggaggatcc

SEQ ID NO: 13 - DNA, 720 bp, synthetic construct
  source 1..720: mol_type = other DNA
gatgtgcagctggtgcagagcggagctgaggtgaagaagccaggagcttctgtgaaggtgtcctgtaaggccagcggctacaccttcacaagatataccatgcactgggtgcgccaggctccaggacagggactggagtggatcggctacatcaacccctctagaggctacaccaattatgccgactccgtgaagggccgctttacaatcaccacagataagagcacctctacagcttacatggagctgtccagcctgaggtccgaggacaccgccacatactattgcgctcggtactatgacgatcattactgtctggattactggggtcagggcaccacagtgaccgtgtcttctggcggcggcggcagtggcggcggcggctccggcggcggcggctccgacatcgtgctgacccagtcccctgccacactgtccctgagcccaggagagagggccaccctgagctgccgggcttctcagtccgtgagctacatgaactggtatcagcagaagccaggcaaggctcccaagagatggatctacgatacatctaaggtggcctccggagtgccagctcgcttctctggctctggaagcggaaccgactatagcctgacaatcaactctctggaggccgaggatgccgctacctactattgtcagcagtggagctctaatcccctgacctttggcggcggcacaaaggtggagatcaag

SEQ ID NO: 14 - protein, 21 aa, synthetic construct
MEHSTFLSGLVLATLLSQVSP

SEQ ID NO: 15 - protein, 330 aa, synthetic construct [sequence omitted]

SEQ ID NO: 16 - protein, 107 aa, synthetic construct [sequence omitted]

SEQ ID NO: 17 - protein, 120 aa, synthetic construct
EVQLVQSGAELVKPGASVKLSCAASGFNIKDTYIYWVRQAPGQGLEWVGRIEPMNGNTKYDPKFQGRFTMTANTSKNTAYLQLSSLRSEDTAVYYCSRGGWLLLYVMDYWGQGTLVTVSS

SEQ ID NO: 18 - protein, 116 aa, synthetic construct
EVQLVESGAGVKQPGGTLRLTCTASGFSLTTYGVHWVRQAPGKGLEWVGVIWGDGRTDYDAAFMSRVTISKDTSKSTVYLQMNSLRAEDTAVYYCMRNRHDWFDYWGQGTTVTVSS

SEQ ID NO: 19 - protein, 112 aa, synthetic construct
DIVMTQSPSSLSVTPGERVSISCRSDKSLLHSNGNTYLYWFLQKPGQSPQVLIYRMSNLASGVPDRFSGSGSGTDFTLTISRVEAEDVGVYYCFQQLEYPYTFGQGTKLEIK

SEQ ID NO: 20 - protein, 107 aa, synthetic construct
DILLTQSPSSLSATPGERATITCRASQGIRNNLNWYQQKPGQAPRLLIYYSSNLQSGVPSRFSGSGSGTDFTLTISRLQPEDVAAYYCQQSIKLPFTFGSGTKVEIK

(19) United States
(12) Patent Application Publication
(10) Pub. No.: US 2026/0217852 A1
(43) Pub. Date: Jul. 30, 2026

(54) ANTI-CD26 ANTIBODY AND USE THEREOF

(71) Applicant: ZONHON BIOPHARMA INSTITUTE, INC., Jiangsu (CN)

(72) Inventors: Bruce Yong MA, Jiangsu (CN); Peipei CAO, Jiangsu (CN); Chennan GE, Jiangsu (CN); Hao YUAN, Jiangsu (CN); Tao ZHANG, Jiangsu (CN)

(21) Appl. No.: 19/149,011

(22) PCT Filed: Dec. 28, 2023
(86) PCT No.: PCT/CN2023/142580

§ 371 Date: Jul. 17, 2025

(30) Foreign Application Priority Data
Jan. 18, 2023 (CN) 202310066264.6

(51) Int. Cl. C07K 16/28; A61K 47/68; A61P 35/00

(52) U.S. Cl. CPC C07K 16/2896; A61K 47/6829; A61K 47/6849; A61P 35/00; C07K 2317/24; C07K 2317/31; C07K 2317/52; C07K 2317/565; C07K 2317/73; C07K 2317/77; C07K 2317/92

(57) ABSTRACT

Provided are an antibody or antigen-binding fragment that binds to human CD26, and use thereof in the preparation of a medicament for treating tumors with high CD26 expression. The mouse-derived anti-CD26 antibody and antigen-binding fragment has high specificity, affinity, and binding activity, as well as cytotoxic activity against various tumor cells. Based on this mouse-derived anti-CD26 antibody, humanized bispecific antibodies, full-length humanized antibodies, and antibody-drug conjugates have been successfully constructed. Compared with other antibodies derived from other mouse antibodies, these antibodies demonstrate higher affinity and binding activity to tumor cells, higher internalization levels in tumor cells, and lower internalization levels in non-tumor cells, thereby exhibiting improved efficacy and safety profiles in ADCs.

### CROSS REFERENCE TO THE RELATED APPLICATIONS

This application is the national phase entry of International Application No. PCT/CN2023/142580, filed on Dec. 28, 2023, which is based upon and claims priority to Chinese Patent Application No. 202310066264.6, filed on Jan. 18, 2023. The entire contents of International Application No. PCT/CN2023/142580 and Chinese Patent Application No. 202310066264.6 are incorporated herein by reference.

### REFERENCE TO AN ELECTRONIC SEQUENCE LISTING

The instant application contains a Sequence Listing which has been submitted in XML format via EFS-Web and is hereby incorporated by reference in its entirety. Said XML copy is named SequenceListing.xml, created on Jul. 16, 2025, and is 21,056 bytes in size.

### TECHNICAL FIELD

The present disclosure relates to the field of biopharmaceutical technology, and specifically to an antibody or antigen-binding fragment that binds to human CD26, and its use in the preparation of medicaments for treating tumors with high CD26 expression.

### BACKGROUND

CD26 is a multifunctional type II transmembrane glycoprotein, and can also exist in a soluble form in plasma. CD26 commonly exists as a homodimer, with each monomer composed of 766 amino acid residues and a molecular mass of approximately 110 kDa. The amino acid residues of CD26 are divided into five parts from the inside and outside: the intracellular region (1-6), the transmembrane region (7-28), the highly glycosylated region (29-323), the cysteine-rich region (324-551) and the C-terminal catalytic domain (552-766). The three-dimensional structure of the molecule is closely related to its function. CD26 (also known as DPP4) inhibitors have been used clinically for the treatment of type II diabetes for several decades. The expression of CD26 on the surface of various tumor cells is significantly increased, such as malignant mesothelioma, renal cancer, prostate cancer, lung cancer, etc. For these types of tumors with high CD26 expression, CD26 is a valuable target (CD26/DPP4—a potential biomarker and target for cancer therapy, Pharmacology & Therapeutics (2019), 198:135-159).

Currently, among anti-cancer therapeutics targeting human CD26, the monoclonal antibody YS110 developed by Y's Therapeutics is progressing most rapidly. Results from the completed Phase II clinical trials of YS110 indicate that the antibody exhibits good tolerability; however, its disease control rate did not meet expectations, which corresponds to the low activity observed in preclinical studies of YS110.

Although the widespread distribution of CD26 has been reported in the literature, the use of YS110 is still relatively safe, indicating that its safety in clinical use is well guaranteed. In a clinical trial involving 30 patients treated with YS110, no deaths were reported. The side effects were mainly infusion-related reaction (16.1%), hiccups (9.7%), diarrhea (6.5%). The serious adverse reactions included two cases of interstitial lung disease, and one case each of pneumonia bacterial, tumor lysis syndrome, hypercapnia, and hypoxia. (Phase 2 Study of YS110, a Recombinant Humanized Anti-CD26 Monoclonal Antibody, in Japanese Patients With Advanced Malignant Pleural Mesothelioma, JTO Clinical and Research Reports (2021), 2 (6))

As described above, although the safety of CD26 as a therapeutic target has been validated in clinical trials, it is difficult for existing CD26-targeting antibodies to achieve good therapeutic effects in the treatment of tumors.

Antibody-drug conjugates (ADCs) are composed of three elements: an antibody, a linker, and a cytotoxic small molecule. ADCs leverage the antibody's specific recognition of antigens to deliver toxin molecules to tumor cells for localization and release drug molecules, thereby achieving the therapeutic purpose of killing tumor cells. Antibody-drug conjugates possess both the antigen-specific recognition ability conferred by the antibody component and tumor cell-killing effect mediated by the toxin molecules. The antibody component of an ADC primarily functions to bind specifically to the target antigen, conferring tumor-targeting properties to the conjugate. To date, 15 ADCs have been approved for marketing worldwide, with the majority targeting HER2.

In general, the antibody component of an ADC should possess characteristics such as high specificity, high affinity, low immunogenicity, high internalization, and long half-life. Currently, no ADCs targeting CD26 have been approved or entered clinical development, and the potential of CD26 as an ADC target remains to be further explored.

### SUMMARY

To address the above-described problems, a primary objective of the present disclosure is to provide an anti-CD26 antibody or antigen-binding fragment having higher specificity, affinity, and better internalization capability.

The antibody or antigen-binding fragment specifically binding to human CD26 provided by the present disclosure comprises an HCDR1 as set forth in SEQ ID NO: 1, an HCDR2 as set forth in SEQ ID NO: 2, and an HCDR3 as set forth in SEQ ID NO: 3; and comprises an LCDR1 as set forth in SEQ ID NO: 4, an LCDR2 having the amino acid sequence of YRS (wherein Y, R, and S represent tyrosine, arginine, and serine, respectively), and an LCDR3 as set forth in SEQ ID NO: 5.

The antibody or antigen-binding fragment described herein comprises a heavy chain variable region having an amino acid sequence with at least 90%, at least 91%, at least 92%, at least 93%, at least 94%, at least 95%, at least 96%, at least 97%, at least 98%, or at least 99% identity to an amino acid sequence as set forth in SEQ ID NO: 8, and a light chain variable region having at least 90%, at least 91%, at least 92%, at least 93%, at least 94%, at least 95%, at least 96%, at least 97%, at least 98%, or at least 99% identity to an amino acid sequence as set forth in SEQ ID NO: 9.

The antibody or antigen-binding fragment described herein, wherein the amino acid sequence set forth in SEQ ID NO: 8 or the amino acid sequence set forth in SEQ ID NO: 9 comprises one, two, three, four, five, six, seven, eight, nine, or ten amino acid insertions, amino acid deletions, or amino acid substitutions, wherein the amino acid substitutions are conservative amino acid substitutions.

The antibody or antigen-binding fragment described herein comprises a heavy chain variable region as set forth in SEQ ID NO: 8 and a light chain variable region as set forth in SEQ ID NO: 9.

The present disclosure further relates to an antibody-drug conjugate (ADC) targeting CD26, comprising the anti-CD26 antibody described above.

The present disclosure also relates to a nucleotide sequence encoding the amino acid sequence of the antibody or antigen-binding fragment described herein.

The present disclosure further relates to a vector comprising the nucleotide sequence and a host cell comprising the nucleic acid.

The present disclosure further relates to a method of producing the antibody or antigen-binding fragment, the method comprising culturing the host cell and recovering the antibody or antigen-binding fragment from the culture.

The present disclosure also relates to a pharmaceutical composition comprising the antibody or antigen-binding fragment described herein, which also comprises a pharmaceutically acceptable excipient.

The present disclosure further relates to a method of treating a tumor, comprising administering an effective amount of the antibody or antigen-binding fragment described herein to a subject. The tumor expresses CD26 at a high level and includes, but is not limited to, renal carcinoma, mesothelioma, lung cancer, liver cancer, and prostate cancer. The antibody or antigen-binding fragment is administered alone or in combination with other anti-cancer agents.

The present disclosure provides the following advantages over the prior art:

The anti-CD26 antibody screened in the present disclosure has high specificity, affinity, and binding activity toward CD26-positive tumor cells. In particular, the full-length humanized antibody derived from mouse antibody No. 79 has higher affinity, binding activity, and internalization efficiency toward tumor cells compared to other full-length humanized antibodies derived from different mouse antibodies, making it more suitable as the antibody component in an antibody-drug conjugate (ADC).

### BRIEF DESCRIPTION OF THE DRAWINGS

FIG. 1 is a flow cytometry analysis showing the differences in antibody binding activity in a mixed cell system.

FIG. 2 is a flow cytometry analysis showing the changes in fluorescence intensity following internalization of the antibody into 786-0 cells.

FIG. 3 is a flow cytometry analysis showing the changes in fluorescence intensity following internalization of the antibody into PBMC.

FIG. 4 is a flow cytometry analysis showing the changes in fluorescence intensity following internalization of the antibody into different PBMCs.

FIG. 5 is a graph showing the cell viability (at 72 h) of UT16 cells induced by DT3C-conjugated 21G428-429, 21G430-431, or YS110.

FIG. 6 is a graph showing the cell viability (at 72 h) of 786-0 cells induced by DT3C-conjugated 21G428-429, 21G430-431, or YS110.

### DETAILED DESCRIPTION OF THE EMBODIMENTS

Unless otherwise defined herein, the technical terms used in the present disclosure shall have the meanings commonly understood by those of ordinary skill in the art.

As used herein, singular forms such as “a,” “an,” and “the” include their plural referents unless the context clearly dictates otherwise.

The term “or” as used herein is intended to mean “and/or” and may be used interchangeably with “and/or.”

The term “CD26” is also known as dipeptidyl peptidase 4 (DPP4). The amino acid sequence of human CD26 is available in the Genbank database under the accession number NP_001926.2, and its cDNA sequence is available in the Genbank database under the accession number NM_001935.3.

The term “conservative amino acid substitution” refers to the replacement of an original amino acid with another amino acid that does not substantially alter the chemical, physical, and/or functional properties of the antibody or fragment, such as its binding affinity for CD26. Conservative substitutions of amino acids are well known in the art. For example, alanine (Ala) can be conservatively substituted with glycine (Gly) or serine (Ser); arginine (Arg) can be substituted with lysine (Lys) or histidine (His); asparagine (Asn) can be substituted with glutamine (Gln) or histidine (His), etc.

The term “affinity” refers to the strength of the interaction between an antibody and an antigen. The variable region of an antibody interacts with an antigen through non-covalent forces; the more interactions, the higher the affinity.

The term “antibody” refers to a member of the immunoglobulin family capable of binding a corresponding antigen in a specific, non-covalent, and reversible manner. For example, a naturally occurring IgG antibody is a tetramer comprising at least two heavy chains and two light chains linked to each other via disulfide bonds. Each heavy chain comprises a heavy chain variable region (VH) and a heavy chain constant region (CH). The heavy chain constant region comprises three domains: CH1, CH2, and CH3. Each light chain comprises a light chain variable region (VL) and a light chain constant region (CL). The light chain constant region comprises a single CL domain. The VH and VL regions can be further subdivided into complementarity-determining regions (CDRs), also referred to as hypervariable regions, and more conserved framework regions (FRs). Each VH and VL comprises three CDRs and four FRs arranged in the following order from the amino terminus to the carboxyl terminus: FR1, CDR1, FR2, CDR2, FR3, CDR3, and FR4. HCDR1, HCDR2, and HCDR3 refer to the three heavy chain complementarity-determining regions; LCDR1, LCDR2, and LCDR3 refer to the three light chain complementarity-determining regions. The positions of the CDRs and FRs can be defined using various numbering schemes known in the art, such as Kabat, Chothia, and IMGT. The present disclosure adopts the IMGT numbering scheme. The variable regions of the heavy chain (VH) and light chain (VL) are responsible for antigen recognition, particularly through their complementarity-determining regions (CDRs), which together typically confer specificity to a particular epitope on the antigen. The constant regions are primarily responsible for effector functions.

The term “antigen-binding fragment” refers to a fragment of an antibody that retains the ability to specifically bind to an antigen, such as a fragment retaining one or more complementarity-determining regions (CDRs), including but not limited to Fab fragments, Fv fragments, bispecific antibodies, linear antibodies, single-chain antibodies, nanobodies, and multispecific antibodies.

The term “monoclonal antibody” refers to a population of antibodies that is essentially homogeneous, meaning that, except for a small number of possible naturally occurring mutations, the antibody molecules within the population have identical amino acid sequences. In contrast, polyclonal antibodies generally comprise a variety of different antibodies with different amino acid sequences in their variable domains, particularly in their complementarity-determining regions (CDRs). Monoclonal antibodies can be obtained using methods known to those skilled in the art. In the present disclosure, monoclonal antibodies are obtained using hybridoma technology, which is one of the known methods of producing monoclonal antibodies.

The term “murine-derived antibody” refers to an antibody that comprises only immunoglobulin sequences derived from mouse or rat.

The term “chimeric antibody” refers to an antibody in which the variable regions are derived from a non-human (e.g., murine) antibody sequence, and the remaining portions are derived from a human antibody sequence.

The term “humanized antibody” refers to an antibody in which the CDRs are derived from a non-human (e.g., murine) antibody sequence, and the remaining portions are derived from a human antibody sequence.

The term “bispecific antibody” refers to an antibody possessing two antigen-binding sites with specificity for the same or different antigens.

The term “BiTE” refers to a type of bispecific antibody, which stands for “bispecific T cell engager.” It is composed of two antigen-specific single-chain variable fragments (scFvs) connected via a linker, wherein one scFv is specific for a tumor-associated surface antigen and the other scFv is specific for CD3 expressed on the surface of T cells. A single-chain variable fragment (scFv), also referred to as a single-chain antibody, is composed of a heavy chain variable region and a light chain variable region connected via a linker. The “linker” refers to an amino acid sequence used to join different protein fragments. Numerous studies have been conducted on the design and selection of linkers. In a BiTE, a linker connects a heavy chain variable region and a light chain variable region to form a scFv, and another linker connects two scFvs in tandem and enables their free rotation. Such linkers are generally composed of repeated GGGGS sequences, where S denotes serine and G denotes glycine. Glycine has a small molecular weight and a short side chain, which increases the flexibility of the side chain, while serine has strong hydrophilicity, which enhances the hydrophilicity of the peptide chain. Optimizing the number of repeat sequences can not only facilitate the correct conformational association between the light chain and heavy chain in the scFv, but also influence the distance between the two scFvs to promote optimal interactions between T cells and target cells at the immunological synapse. (Zhou Chong et al., Research Progress on Bispecific Single-Chain Antibody BiTE [J], Journal of Biology, 2018). Linkers commonly used to connect the heavy chain variable region and the light chain variable region in scFvs include, for example, KESGSVSSEQLAQFRSLD, EGKSSGSGSESKST, GSTSGGGSGGGSGGGGSS (US20180326032), and GSTSGSGKPGSGEGSTKG (Preclinical Development of Bivalent Chimeric Antigen Receptors Targeting Both CD19 and CD22). In the specific embodiments of the present disclosure, the flexible linker connecting a heavy chain and a light chain to form an scFv comprises three repeats of the GGGGS motif, and the flexible linker connecting two scFvs in tandem comprises a single GGGGS motif. One of ordinary skill in the art can select and determine linkers from known linkers based on the teachings of the prior art through routine experimentation and a limited number of experiments. The number of GGGGS repeats in the two types of flexible linkers can be optimized, and flexible linkers other than GGGGS can also be used.

CD3 is a component of T cell signaling. When a BiTE molecule simultaneously binds to a T cell and a tumor cell, the T cell is activated, promoting CD8+ T cells to directly secrete perforin and granzyme, and CD4+ T cells to secrete cytokines that further recruit and activate cytotoxic T cells, thereby killing tumor cells. The anti-CD3 antibody sequence can be selected from OKT-3, L2K, TR66, UCHT1, SP34, IORT3, Catumaxomab, Blinatumomab, Solitomab, and other anti-CD3 antibody sequences known in the art. In the specific embodiments of the present disclosure, the anti-CD3 antibody sequence from Solitomab is used. A person skilled in the art can select and determine other known anti-CD3 antibody sequences through routine experimentation and a limited number of experiments, and is not limited to the specific anti-CD3 antibody sequence exemplified herein.

The term “antibody-drug conjugate” (ADC) refers to a construct in which a small-molecule toxin is conjugated to an antibody via a linker, wherein the antibody functions as a carrier to deliver the small-molecule toxin to target cells in a targeted manner.

The terms “administration” or “administering”, and “treatment” or “treating”, refer to the contact of an exogenous agent, therapeutic agent, or diagnostic agent with the tissues, organs, cells, or biological fluids of a subject. The term “treatment” or “treating” refers to retarding or preventing the development of clinical symptoms of a disease, alleviating or improving at least one physical parameter, or preventing the progression of the disease.

### Example 1. Analysis of D26-Positive and CD26-Negative Cells

Target cells were cultured in T75 cell culture flasks. When the cells reached over 80% confluence, they were digested with trypsin and collected. The cells were washed once with PBS, counted using a hemocytometer, and aliquoted into portions containing 5×10⁵ cells each. A monoclonal anti-CD26 antibody was used as the primary antibody and incubated with the target cells at room temperature for approximately 40 minutes. After incubation, the cells were centrifuged, and the supernatant was discarded. The cell pellet was resuspended in PBS, followed by centrifugation and discard the supernatant again, and the cell pellet was collected. Subsequently, an Alexa Fluor 488-conjugated mouse anti-human IgG1 antibody was used as the secondary antibody. The cell pellet was resuspended and incubated with the secondary antibody at room temperature for approximately 30 minutes in the dark. After incubation, the cells were washed twice with PBS, centrifuged and discard the supernatant, and the cell pellet was collected. The cell pellet was resuspended in approximately 200 μL of PBS solution and analyzed by flow cytometry within 1 hour to determine the percentage of CD26-positive cells.

The expression of CD26 was analyzed by flow cytometry. The cell lines 786-0, OS-RC-2, UT16, and NCI-H226 were identified as CD26-positive, while G401 and A-375 were identified as CD26-negative. The percentage of CD26-positive cells is shown in Table 1. The results for the CD26-positive cell lines are consistent with those reported in the literature (Chinese Patent Application No. CN200680034937.4; CD26/DPP4-a potential biomarker and target for cancer therapy, Pharmacology & Therapeutics (2019), 198:135-159).

TABLE 1
CD26 Positivity Rate of Target Cells

| | | | CD26 positivity |
|---|---|---|---|
| NO. | Cell line name | Tumor type | rate |
| 1 | 786-0 | Kidney cancer | 99.9% |
| 2 | OS-RC-2 | | 97.8% |
| 3 | G401 | | 0% |
| 4 | UT16 | | 99.5% |
| 5 | NCI-H226 | Mesothelioma | 99.4% |
| 6 | A-375 | Melanoma | 2.90% |

### Example 2 Preparation and Screening of Anti-Human CD26 Hybridoma Cell Lines

### Step 1: Animal Immunization

Based on the publicly available cDNA sequence of CD26 (GenBank accession number: NM_001935.3), an rCD26 expression vector was designed. A histidine tag was introduced at the 5′ end of the codon sequence, and the full-length gene was synthesized. The synthesized gene was cloned into the pCHO1.0 plasmid, expressed in CHO-S host cells, and subsequently purified. The prepared recombinant CD26 target protein was used to immunize female BALB/c mice following routine immunization procedures. Detailed immunization protocols are described in “MAKING and USING ANTIBODIES”. Indirect ELISA was employed to monitor the serum titers of immunized mice. The mouse exhibiting the highest serum titer was selected, and a cell fusion experiment was conducted using the mouse's spleen cells and myeloma cells.

### Step 2: Cell Fusion

### (1) Preparation of Spleen Cells

The immunized mouse was subjected to orbital blood collection, sacrificed by cervical dislocation, and immersed in 75% (v/v) ethanol for 10 minutes. The spleen was aseptically harvested on a sterile workbench, placed on a cell strainer, and thoroughly mashed, which were then passed through the cell strainer. The cells were washed several times by centrifugation using sterile 1640 medium (purchased from Gibco), and subsequently resuspended to obtain a single-cell suspension. The cells were counted and stored for subsequent use.

### (2) Preparation of Feeder Cells

A female BALB/c mouse aged 8-10 weeks was selected. Orbital blood was collected to obtain negative serum. The mouse was sacrificed by cervical dislocation and then immersed in 75% (v/v) ethanol for 10 minutes. Under aseptic conditions, the abdominal skin was aseptically opened to expose the peritoneum. Approximately 10 mL of 1640HT medium (purchased from SIGMA) was injected into the peritoneal cavity using a syringe. The abdomen was gently massaged, and the fluid was pipetted up and down several times. The medium containing macrophages was aspirated and added to 20% 1640HAT medium for later use.

A female BALB/c mouse aged 2-3 weeks was selected. Sacrificed by cervical dislocation and then immersed in 75% (v/v) ethanol for 10 minutes. Under aseptic conditions, the thymus was aseptically harvested and placed on a cell strainer, mashed and passed through the cell strainer. The resulting thymocytes were added into the above 20% 1640HAT medium containing macrophages for subsequent use.

### (3) Cell Fusion

Mouse myeloma cell line SP2/0 in logarithmic growth phase were collected and counted. Approximately 1×10⁸ of the above spleen cells and 2×10⁷ of the above SP2/0 cell line were added to a fusion tube and mixed, then centrifuged at 1,000 rpm for 10 minutes. The supernatant was discarded, and the fusion tube was placed on the palm and gently rubbed back and forth to loosen the pellet. Within 60 seconds, 1 mL of pre-warmed PEG 1450 (polyethylene glycol 1450; purchased from Sigma) was added slowly at first and then gradually faster, followed by addition of 30 mL of 1640HT medium to terminate. Centrifuged at 1,000 rpm for 10 minutes; the supernatant was discarded; the pellet was loosened by rubbing gently and added to the 20% 1640HAT medium obtained in Step 2.

After thorough mixing, the above HAT medium was dispensed into a 96-well cell culture plate at 200 μL per well and incubated at 37° C. in a 5% CO₂ incubator. After one week, the 20% 1640HAT medium was replaced with 10% 1640HT medium, and the supernatant was collected for assay three days later.

### Step 3: Screening of Hybridoma Cell Lines Specific for CD26

(1) Preparation of ELISA plate: The recombinant CD26 target protein was diluted to 1 μg/mL using CB coating buffer and used to coat a 96-well ELISA plate at 100 μL per well. The plate was incubated at 2-8° C. overnight. After washing once, the plate was tapped dry. The wells were blocked with PBST buffer containing 2% BSA (200 μL per well) at 37° C. for 2 hours. The plate was then tapped dry and kept for later use.
(2) Screening of positive clones: The culture supernatant of the cells to be tested was added to the prepared ELISA plate (100 μL per well) and incubated at 37° C. for 30 minutes. After washing, the plate was tapped dry. Diluted HRP-conjugated goat anti-mouse IgG was added at 100 μL per well and incubated at 37° C. for 30 minutes. After washing, the plate was tapped dry. TMB chromogenic solution was added at 100 μL per well and incubated at 37° C. in the dark for 15 minutes. Then, 50 μL per well of 2 M H₂SO₄ was added to terminate the reaction. The absorbance at 450 nm (OD₄₅₀) was measured. A well was determined to be positive if the (OD₄₅₀ value/negative control value) was ≥2.1. Positive clones were selected for clonal screening. After three to four rounds of clonal screening, the monoclonal cell line with a 100% positivity rate was confirmed as a stable cell line, and the cell line was established.

### Step 4: Activity Evaluation of Mouse Anti-CD26 Monoclonal Antibody (Mouse Monoclonal Antibody No. 79)

Tumor cells were labeled with the Calcein-AM fluorescent dye to exhibit green fluorescence signal. The tumor cells were seeded into a U-bottom 96-well cell culture plate at a concentration of 6×10⁵ cells/mL, 50 μL per well. Three replicate wells were set for each group. 50 μL of anti-CD26 antibody was added to the corresponding sample wells to reach a final concentration of 100 ng/mL. For the blank control wells, 50 μL of culture medium was added, and for the positive control wells, 50 μL of Triton X-100 was added at a final concentration of 1%. The plate was incubated at 37° C. for 30 minutes. Subsequently, PBMCs (peripheral blood mononuclear cells) at a concentration of 6×10⁶ cells/mL were added at an effector-to-target (E:T) ratio of 10:1. The reaction system was incubated at 37° C. under CO₂ atmosphere for 5 hours. After incubation, the plate was centrifuged and the supernatant was collected and transferred to a new 96-well plate. After a second centrifugation, 80 μL of supernatant was collected and transferred to a black 96-well plate, and fluorescence intensity was measured using a microplate reader at an excitation wavelength of 470 nm and an emission wavelength of 515 nm.

The formula for calculating the cell lysis rate was as follows:

$$Cell lysis rate ( \% ) = [ \text{} ( {V}_{sample} - {V}_{vehicle control} ) / ( {V}_{Triton X - 100} - {V}_{vehicle control} ) ] \times 100 \%$$

Where Vₛₐₘₚₗₑ is the mean value of the fluorescence signal reading of the drug-treated group measured at the specified excitation and emission wavelengths; V_{vehicle} control is the mean value of the fluorescence signal reading of the blank control group measured at the same excitation and emission wavelengths; and V_{Triton X-100} is the mean value of the fluorescence signal reading of the positive control group measured at the same excitation and emission wavelengths.

TABLE 2
The cytotoxic effect of PBMCs on target
cells mediated by mouse antibody No. 79

| | cell line name | | |
|---|---|---|---|
| | 786-0 | OS-RC-2 | NCI-H226 |
| cell lysis rate | 48.8% | 34.4% | 25.5% |

From the above results, under the conditions of an antibody concentration of 100 ng/ml and an incubation time of 5 hours, the cell lysis rate of target cells 786-0 induced by PBMCs mediated by mouse antibody No. 79 was 48.8%. Under the same conditions, the cell lysis rate of target cells 786-0 induced by PBMCs mediated by other mouse antibodies (Nos. 62, 72, 160, 51, and 212) were 53.1%, 8.6%, 13.0%, 12.3%, and 11.3%, respectively. The cell lysis rate of target cells OS-RC-2 induced by PBMCs mediated by mouse antibody No. 79 was 34.4%. Under the same conditions, the cell lysis rate of target cells OS-RC-2 induced by PBMCs mediated by other mouse antibodies (Nos. 47, 76, 62, and 82) were 15.4%, 19.9%, 49.9%, and 30.8%, respectively. The cell lysis rate of target cells NCI-H226 induced by PBMCs mediated by mouse antibody No. 79 was 25.5%. Under the same conditions, the cell lysis rate of target cells NCI-H226 induced by PBMCs mediated by other mouse antibody (Nos. 47, 51, 62, and 82) were 6.7%, 21.0%, 14.3%, and 17.5%, respectively. These results indicate that mouse antibody No. 79 has cytotoxic activity against different CD26-positive cell lines.

### Example 3. Screening and Sequencing of Variable Region Sequences from Hybridoma Cell Lines

Step 1. Total RNA Extraction from CD26-Specific Hybridoma Cells

The hybridoma cells were passaged into T75 culture flasks and cultured until approximately 90% confluence. The cells were then digested and collected by centrifugation. Total RNA was extracted from the monoclonal hybridoma cell lines using an RNA extraction kit (purchased from Roche). The extracted total RNA was used as a template for synthesizing the first-strand cDNA via reverse transcription using a cDNA synthesis kit (purchased from Thermo). The reaction products were stored at −20° C.; for long-term storage, were stored at −70° C.

### Step 2. PCR Amplification of the Heavy and Light Chain Variable Region Genes

The first-strand cDNA of the hybridoma cells was used as a template in a 50 μL reaction system, which contained: 1 μL of cDNA, 5 μL of 10×PCR buffer, 1 μL each of forward and reverse primers (25 μmol), 1 μL of dNTPs, 1 μL of 25 mmol/L MgCl₂, and 39 μL of H₂O. After the initial denaturation at 95° C. for 10 minutes, 1 μL of Taq polymerase was added to initiate thermal cycling for PCR amplification. The reaction conditions were as follows: denaturation at 94° C. for 1 minute, annealing at 58° C. for 1 minute, and extension at 72° C. for 1.5 minutes, for a total of 30 cycles, followed by holding at 72° C. for 10 minutes. 5 μL of the PCR product was analyzed by 1.2% agarose gel electrophoresis.

Step 3. Cloning and sequencing of the heavy and light chain variable region genes According to the instructions of the pGM-T Fast Ligation Kit (VT207-02, Beijing Tiangen Biotech Co., Ltd.), the variable region genes of the heavy and light chains were respectively ligated into the pGM-T vector and transformed into Escherichia coli Top10 competent cells. Blue-white screening was performed, and the transformants were cultured at 37° C. for 12-16 hours.

The resulting white colonies were inoculated into 1-5 mL of LB medium containing ampicillin at a final concentration of 100 μM, and shaken at 37° C. for 3-4 hours. PCR was performed to identify clones carrying the correct insert. The positive clones identified by PCR screening were subjected to sequencing. The sequencing results were analyzed by comparison with the IMGT database to determine the amino acid sequences of the variable regions and complementarity-determining regions (CDRs) of the heavy and light chains of the antibody.

The No. 79 mouse monoclonal antibody comprises an HCDR1 as shown in SEQ ID NO: 1, an HCDR2 as shown in SEQ ID NO: 2, and an HCDR3 as shown in SEQ ID NO: 3; and comprises an LCDR1 as shown in SEQ ID NO: 4; an LCDR2 with the amino acid sequence YRS; and LCDR3 as shown in SEQ ID NO: 5. The heavy chain variable region is shown in SEQ ID NO: 6, and the light chain variable region is shown in SEQ ID NO: 7.

### Example 4: Construction of Humanized Bispecific Antibody

The amino acid sequences of the humanized heavy and light chain variable regions of mouse antibody No. 79 are as follows: 19G300-VH (SEQ ID NO: 8) and 19G300-VL (SEQ ID NO: 9).

The above humanized heavy and light chain variable regions were linked via a short peptide linker (SEQ ID NO: 10), and an additional short peptide linker (SEQ ID NO: 11) was added downstream. The resulting sequence was codon-optimized based on the codon preference of CHO (Chinese Hamster Ovary) cells to obtain an optimized anti-CD26 single-chain antibody gene sequence (SEQ ID NO: 12). An AvrII restriction site and a Kozak sequence were introduced upstream, and an anti-CD3 single-chain antibody gene (SEQ ID NO: 13), a stop codon, and a BstZ17I restriction site were added downstream. The resulting humanized CD26-CD3 BiTE gene sequence was directly synthesized and cloned into the pUC57 plasmid, designated as pUC57-19G300.

The target gene was amplified, and the PCR product was recovered by 1% agarose gel electrophoresis. The recovered PCR product and the pZHK2.0 vector were double-digested with AvrII and BstZ17I. The double-digested product was ligated into the pZHK2.0 vector using T4 DNA ligase, followed by transformation into Escherichia coli Top10 competent cells. The transformed cells were plated onto LB plates containing kanamycin and incubated overnight at 37° C. On the following day, positive clones were screened and subjected to sequencing analysis. The sequence was confirmed to be completely identical to the expected sequence, indicating that the humanized CD26-CD3 BiTE bispecific antibody expression plasmid was successfully constructed.

A mammalian cell line capable of stably expressing the humanized CD26-CD3 BiTE bispecific antibody at high levels was inoculated into Dynamis medium and cultured under fed-batch conditions at 37° C., 8% CO₂, and 130 rpm. The culture supernatant was harvested, and centrifuged at 12,000 rpm for 15 minutes at a low temperature to collect the supernatant. The collected supernatant was filtered through a 0.45 μm membrane to obtain the processed culture supernatant, which was then subjected to chromatographic purification. The molecular weight of the final product was approximately 55 kDa.

### Example 5: Affinity Analysis of Humanized Bispecific Antibody for CD26 Protein and CD3 Protein

### 1. Affinity Analysis of the Antibody for CD26 Protein

CD26 protein was first biotinylated using EZ-Link NHS-PEG12-Biotin, and the biotinylated protein was immobilized on SA biosensors. The biosensors were then equilibrated in 1× Kinetics Buffer, followed by incubation with the test antibody solution to allow binding. The concentration of the antibody solution to be tested was 500 nM. Afterward, the biosensors were placed in 1× Kinetics Buffer for dissociation. Data were analyzed using Fortebio Data Analysis 8.0 software to calculate the affinity constant values.

TABLE 3
affinity of the humanized bispecific
antibody 19G300 for CD26 Protein

| | Sample Name | | |
|---|---|---|---|
| | KD(M) | kon ( 1/Ms ) | kdis(1/s) |
| 19G300 | 1.14E−09 | 5.68E+04 | 6.49E−05 |

### 2. Affinity Analysis of the Antibody for CD3 Protein

CD3 protein was first biotinylated using EZ-Link NHS-PEG12-Biotin, and the biotinylated protein was immobilized on SA biosensors. The biosensors were then equilibrated in 1× Kinetics Buffer, followed by incubation with the humanized bispecific antibody solution to allow binding. The concentration of the humanized bispecific antibody solution was 500 nM. Afterward, the biosensors were placed in 1× Kinetics Buffer for dissociation. Data were analyzed using Fortebio Data Analysis 8.0 software to calculate the affinity constant values.

TABLE 4
affinity of the humanized bispecific
antibody 19G300 for CD3 Protein

| | Sample Name | | |
|---|---|---|---|
| | KD(M) | kon ( 1/Ms ) | kdis(1/s) |
| 19G300 | 1.61E−09 | 8.48E+04 | 1.37E−04 |

In summary, the affinity of 19G300 for CD26 protein was 1.14E-09 M, and the affinity for CD3 protein was 1.61E-09 M.

### Example 6: Construction of Full-Length Humanized Antibody

21G430-431 is a fully humanized anti-CD26 monoclonal antibody derived from mouse antibody No. 79, with its heavy and light chain variable region amino acid sequences designated as 19G300-VH (SEQ ID NO: 8) and 19G300-VL (SEQ ID NO: 9), respectively. 21G428-429 is a fully humanized anti-CD26 monoclonal antibody derived from mouse antibody No. 62, with its heavy and light chain variable region amino acid sequences designated as 19G294-VH (SEQ ID NO: 17) and 19G294-VL (SEQ ID NO: 19), respectively. The heavy and light chain variable region amino acid sequences of YS110 are designated as YS110-VH (SEQ ID NO: 18) and YS110-VL (SEQ ID NO: 20), respectively.

The signal peptide sequence (SEQ ID NO: 14) was added upstream of 19G300-VH (SEQ ID NO: 8), 19G294-VH (SEQ ID NO: 17), and YS110-VH (SEQ ID NO: 18), and the human IgG1 heavy chain constant region (SEQ ID NO: 15) was added downstream of each, thereby constructing the IgG1 heavy chain sequences of the full-length humanized anti-CD26 antibodies 21G430-431, 21G428-429, and YS110. The sequences were codon-optimized based on the codon usage preference of mammalian CHO cells to generate optimized gene sequences. An AvrII restriction site and a Kozak sequence were introduced upstream of the optimized gene sequences respectively, and a stop codon and a BstZ17I restriction site were introduced downstream of each. The genes were directly synthesized and cloned into the pCHO1.0 vector between the AvrII and BstZ17I sites.

A signal peptide sequence (SEQ ID NO: 14) was added upstream of 19G300-VL (SEQ ID NO: 9), 19G294-VL (SEQ ID NO: 19), and YS110-VL (SEQ ID NO: 20), and a human kappa light chain constant region (SEQ ID NO: 16) was added downstream of each, thereby constructing the IgG1 light chain sequences of the full-length humanized anti-CD26 antibodies 21G430-431, 21G428-429, and YS110. The sequences were codon-optimized based on the codon usage preference of mammalian CHO cells to generate optimized gene sequences. An EcoRV restriction site and a Kozak sequence were introduced upstream of the optimized gene sequences respectively, and a stop codon and a PacI restriction site were introduced downstream of each. The genes were directly synthesized and cloned into the pZHK5.18 vector between the EcoRV and PacI sites.

The pCHO1.0 vector (available from Thermo) contained a puromycin resistance gene. The pZHK5.18 vector was derived from the pCHO1.0 by replacing the puromycin resistance gene with a hygromycin resistance gene.

The heavy chain and light chain vectors were co-electroporated into CHO-S cells. Stable CHO cell lines expressing full-length antibodies were obtained by dual selection with hygromycin and puromycin. The stable CHO cell lines were seeded into Dynamis medium and subjected to fed-batch culture at 37° C., 8% CO₂, and 130 rpm.

The culture supernatant was harvested, and centrifuged at 12,000 rpm for 15 minutes at a low temperature to collect the supernatant. The collected supernatant was filtered through a 0.45 μm membrane to obtain the processed culture supernatant. Purification was performed using Cytiva MabSelect Sure affinity chromatography resin. After adjusting the pH of the purified product to 5.5, further purification was performed using Cytiva SP HP cation exchange chromatography resin. The molecular weight of the final products were approximately 150 kDa.

### Example 6: Differences in Binding Ability of Full-Length Humanized Antibodies in a Mixed Cell System

786-0 cells were collected and counted, and the cell density was adjusted to 6×10⁵ cells/mL. PBMCs were collected and counted, and the cell density was adjusted to 6×10⁵ cells/mL. 50 μL each of 786-0 cells and PBMCs were added to an EP tube to form a mixed cell system. The antibody was added to the mixed cell system and diluted therein to a final concentration of 0.3 μg/mL. The mixture was mixed to homogeneity and incubated at 4° C. for 1 hour.

After incubation, the cells were collected by centrifugation, washed twice with PBS, and resuspended in PBS. Goat anti-human IgG fluorescent secondary antibody was added to the cells, followed by incubation at 4° C. for 1 hour. After incubation, the cells were washed twice with PBS and resuspended in PBS. Flow cytometric analysis was performed using a BD ACCURI C6 flow cytometer.

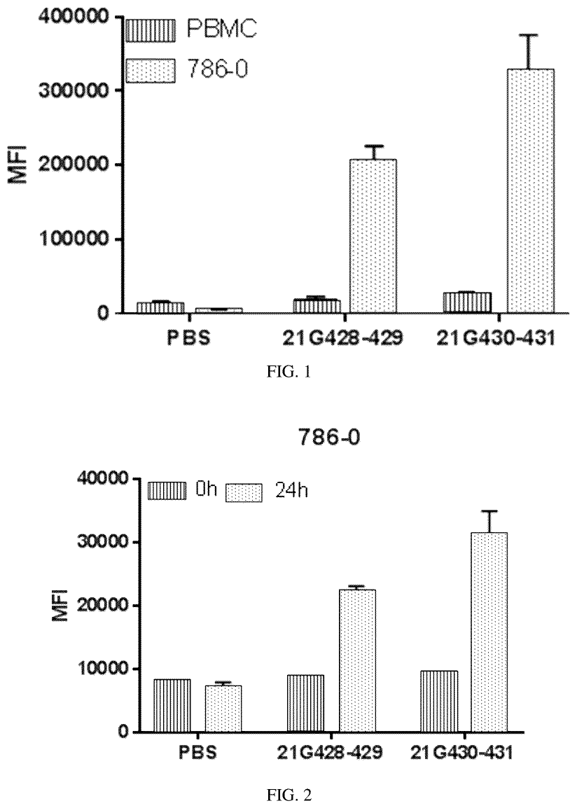

The results (FIG. 1) showed that the full-length humanized anti-CD26 antibody 21G430-431 exhibited significantly higher binding to cancer cells 786-0 compared to the full-length humanized anti-CD26 antibody 21G428-429. In contrast, 21G430-431 and 21G428-429 showed comparable binding to PBMCs. These findings indicate that the full-length humanized antibodies 21G428-429 and 21G430-431 recognize the CD26 target on tumor cells with higher selectivity than on PBMCs, preferentially binding to the CD26 target on tumor cells and exhibiting very low binding to CD26 on PBMCs, thereby avoiding cytotoxicity to normal cells. Furthermore, these findings indicate that the full-length humanized antibody 21G430-431 has a higher binding ability to tumor cells than 21G428-429.

### Example 7: Comparison of Internalization Levels of Full-Length Humanized Antibodies

Antibody-drug conjugates (ADCs) are formed by conjugating antibodies with cytotoxic molecules, combining the target specificity of antibodies with the potent tumor-killing effects of toxins. The antibody component of an ADC is expected to: (1) possess high tumor tissue specificity and strong affinity and binding ability to ensure efficient tumor cell targeting and reduce off-target toxicity; (2) be effectively internalized into tumor cells upon binding to the target antigen, enabling the rapid release of the cytotoxic payload within endosomes or lysosomes, thereby exerting cytotoxic effects through the released small molecule drug.

To evaluate the internalization of antibodies into lysosomes, antibodies were fluorescently labeled using pHrodo iFL Green STP ester in accordance with the manufacturer's instructions. This fluorescent label is pH-sensitive and exhibits significantly enhanced fluorescence under low pH conditions. Since the pH of the lysosomal environment is approximately 5.0, significantly lower than the cytoplasmic pH of 7.2, when the fluorescently labeled antibody enters tumor cells and is trafficked into lysosomes, the significant pH shift from 7.4 to 5.0 leads to a marked increase in fluorescence intensity, which serves as an indicator of whether the antibody has been internalized into lysosomes.

786-0 cells and PBMCs were collected and counted, and the cell density of both was adjusted to 2×10⁵ cells/mL. 50 μL of each cell type were added into an EP tube to create a mixed cell system. The fluorescently labeled antibody was added to the mixed-cell system, diluted to a final concentration of 5 g/mL, thoroughly mixed, and then incubated at 37° C. for 24 hours. Another set of cells, without fluorescently labeled antibody, was prepared and mixed immediately before analysis to serve as the 0-hour control. Before flow cytometric analysis, the samples were centrifuged to remove serum-containing medium and the medium was replaced with PBS. The mean fluorescence intensity was then measured using flow cytometry.

The results (FIG. 2) showed that the full-length humanized anti-CD26 antibody 21G430-431 exhibited significantly higher internalization into 786-0 cells than 21G428-429, indicating that 21G430-431 is more suitable as the antibody component of an ADC.

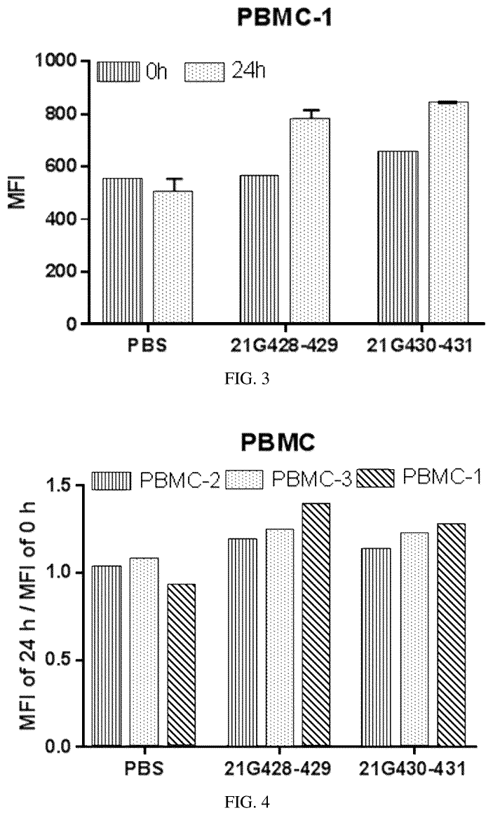

To minimize off-target effects of ADCs, it is desirable for the antibody to have low internalization levels in non-target cells, thereby reducing the risk of adverse effects. As shown in FIG. 3, both anti-CD26 antibodies exhibited lower internalization into PBMCs than into cancer cells. The difference in mean fluorescence intensity between the 24-hour and 0-hour time points indicated that antibody 21G430-431 exhibited slightly lower internalization into PBMCs compared to 21G428-429.

As shown in FIG. 4, internalization assays using PBMCs from three different individuals consistently demonstrated that 21G430-431 exhibited lower internalization levels than 21G428-429.

### Example 8: Cytotoxicity of Anti-CD26 Antibodies Conjugated with Biological Toxin Toward Cancer Cells

According to Examples 7 and 8, antibody 21G430-431 exhibited superior affinity, binding ability, and internalization level compared to 21G428-429, indicating its potential use as the antibody component of an ADC. In the present example, DT3C (diphtheria toxin) was used as a model protein that can bind to the Fc region of an antibody, and after internalization, is cleaved by intracellular proteases to release diphtheria toxin. DT3C was incubated with anti-CD26 antibodies 21G430-431, 21G428-429, and YS110 to construct simplified antibody-drug conjugates, and the cytotoxicity of the antibodies as drug conjugates was preliminarily evaluated.

DT3C and each antibody were added to the culture medium to prepare a solution containing 40 μg/mL DT3C and 20 μg/mL antibody. After mixing thoroughly, the solution was incubated in an incubator for one hour. During the incubation, 786-0 cells and UT16 cells were collected, counted, and the cell density was adjusted to 5×10⁴ cells/mL. Cells were added into 96-well plates at a volume of 50 μL per well. After the incubation of DT3C (40 μg/mL) and antibody (20 μg/mL) solution was completed, 50 μL of the solution was added to each well, and incubation was continued at 37° C. for 72 hours. At the end of incubation, 10 μL of CCK8 solution was added to each well, followed by incubation in the incubator for one hour. Absorbance at 450 nm was measured using a microplate reader. The absorbance value of each sample was corrected by subtracting the background signal of the culture medium without cells. Cell viability was calculated using the group without antibody as the control. The calculation formula was as follows:

$$( {A}_{Sample} - {A}_{Medium} ) / {A}_{Blank}$$

where Aₛₐₘₚₗₑ refers to the absorbance at 450 nm of wells containing cells incubated with the sample,
A_{Medium} refers to the absorbance at 450 nm of wells containing only medium, and
A_{Blank} refers to the absorbance at 450 nm of wells containing cells without antibody solution.

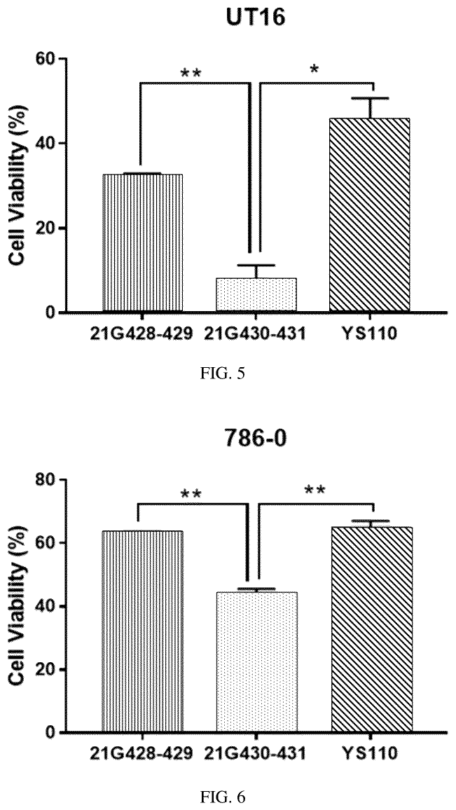

The results (FIG. 5 and FIG. 6) showed that all three anti-CD26 antibody-conjugated drug samples demonstrated a certain level of cytotoxicity toward target cells. The DT3C-conjugated 21G430-431 demonstrated higher cytotoxicity than DT3C-conjugated 21G428-429, with a statistically significant difference. This indicates that an antibody with higher cell affinity, binding ability, and stronger internalization level, when conjugated with toxin molecules, exhibits higher cytotoxicity against target cells. In the present example, YS110, which has progressed to Phase II clinical trials, was also used as a control antibody. Notably, 21G430-431 exhibited stronger ADC efficacy than YS110, with a statistically significant difference.

Based on the results of the above examples, the anti-CD26 antibody derived from mouse antibody No. 79 exhibited high specificity, affinity, and binding ability toward CD26-positive tumor cells. Furthermore, the full-length humanized antibody derived from mouse No. 79 exhibited greater tumor cell affinity, stronger binding ability, enhanced internalization into tumor cells, and lower internalization into non-tumor cells compared to full-length humanized antibodies derived from other mouse antibodies. As a result, it demonstrates superior efficacy and safety as the antibody component in ADCs, and can be used as the antibody component of an ADC.

1. An antibody or antigen-binding fragment that specifically binds to human CD26, comprising an HCDR1 set forth in SEQ ID NO: 1, an HCDR2 set forth in SEQ ID NO: 2, and an HCDR3 set forth in SEQ ID NO: 3, and comprising an LCDR1 set forth in SEQ ID NO: 4, an LCDR2, and an LCDR3 set forth in SEQ ID NO: 5, wherein the LCDR2 has an amino acid sequence of YRS.

2. The antibody or antigen-binding fragment according to claim 1, comprising a heavy chain variable region having an amino acid sequence with at least 90% identity to an amino acid sequence set forth in SEQ ID NO: 8, and a light chain variable region having an amino acid sequence with at least 90% identity to an amino acid sequence set forth in SEQ ID NO: 9.

3. The antibody or antigen-binding fragment according to claim 2, wherein the amino acid sequence set forth in SEQ ID NO: 8 or the amino acid sequence set forth in SEQ ID NO: 9 comprises between one and ten amino acid insertions, amino acid deletions, or amino acid substitutions, wherein the amino acid substitutions are conservative amino acid substitutions.

4. The antibody or antigen-binding fragment according to claim 2, comprising a heavy chain variable region set forth in SEQ ID NO: 8 and a light chain variable region set forth in SEQ ID NO: 9.

5. An antibody-drug conjugate targeting CD26, comprising the antibody or antigen-binding fragment according to claim 1.

6. A nucleotide sequence encoding an amino acid sequence of the antibody or antigen-binding fragment according to claim 1.

7. A vector, comprising the nucleotide sequence according to claim 6.

8. A host cell, comprising the vector according to claim 7.

9. A pharmaceutical composition, comprising the antibody or antigen-binding fragment according to claim 1.

10. A method of treating a tumor with high CD26 expression, comprising administering to a subject an effective amount of the antibody or antigen-binding fragment according to claim 1, or an antibody-drug conjugate targeting CD26 comprising the antibody or antigen-binding fragment, wherein the tumor with high CD26 expression is selected from the group consisting of renal cancer, mesothelioma, lung cancer, liver cancer, and prostate cancer.

11. The antibody-drug conjugate targeting CD26 according to claim 5, wherein the antibody or antigen-binding fragment comprises a heavy chain variable region having an amino acid sequence with at least 90% identity to an amino acid sequence set forth in SEQ ID NO: 8, and a light chain variable region having an amino acid sequence with at least 90% identity to an amino acid sequence set forth in SEQ ID NO: 9.

12. The antibody-drug conjugate targeting CD26 according to claim 11, wherein the amino acid sequence set forth in SEQ ID NO: 8 or the amino acid sequence set forth in SEQ ID NO: 9 comprises between one and ten amino acid insertions, amino acid deletions, or amino acid substitutions, wherein the amino acid substitutions are conservative amino acid substitutions.

13. The antibody-drug conjugate targeting CD26 according to claim 11, wherein the antibody or antigen-binding fragment comprises a heavy chain variable region set forth in SEQ ID NO: 8 and a light chain variable region set forth in SEQ ID NO: 9.

14. The nucleotide sequence according to claim 6, wherein the antibody or antigen-binding fragment comprises a heavy chain variable region having an amino acid sequence with at least 90% identity to an amino acid sequence set forth in SEQ ID NO: 8, and a light chain variable region having an amino acid sequence with at least 90% identity to an amino acid sequence set forth in SEQ ID NO: 9.

15. The nucleotide sequence according to claim 14, wherein the amino acid sequence set forth in SEQ ID NO: 8 or the amino acid sequence set forth in SEQ ID NO: 9 comprises between one and ten amino acid insertions, amino acid deletions, or amino acid substitutions, wherein the amino acid substitutions are conservative amino acid substitutions.

16. The nucleotide sequence according to claim 14, wherein the antibody or antigen-binding fragment comprises a heavy chain variable region set forth in SEQ ID NO: 8 and a light chain variable region set forth in SEQ ID NO: 9.

17. The pharmaceutical composition according to claim 9, wherein the antibody or antigen-binding fragment comprises a heavy chain variable region having an amino acid sequence with at least 90% identity to an amino acid sequence set forth in SEQ ID NO: 8, and a light chain variable region having an amino acid sequence with at least 90% identity to an amino acid sequence set forth in SEQ ID NO: 9.

18. The pharmaceutical composition according to claim 17, wherein the amino acid sequence set forth in SEQ ID NO: 8 or the amino acid sequence set forth in SEQ ID NO: 9 comprises between one and ten amino acid insertions, amino acid deletions, or amino acid substitutions, wherein the amino acid substitutions are conservative amino acid substitutions.

19. The pharmaceutical composition according to claim 17, wherein the antibody or antigen-binding fragment comprises a heavy chain variable region set forth in SEQ ID NO: 8 and a light chain variable region set forth in SEQ ID NO: 9.

20. The method according to claim 10, wherein the antibody or antigen-binding fragment comprises a heavy chain variable region having an amino acid sequence with at least 90% identity to an amino acid sequence set forth in SEQ ID NO: 8, and a light chain variable region having an amino acid sequence with at least 90% identity to an amino acid sequence set forth in SEQ ID NO: 9.
